(19) United States
(12) Patent Application Publication
(10) Pub. No.: US 2018/0120933 A1
(43) Pub. Date: May 3, 2018

(54) IMAGE-BASED SYSTEM, HAVING INTERCHANGEABLE VISION ELEMENTS, TO OBSERVE AND DOCUMENT EYE RESPONSES

(71) Applicant: Ocular Data Systems, Inc., Pasadena, CA (US)

(72) Inventors: Ronald Alan Waldorf, Los Angeles, CA (US); Marcus Orest Filipovich, Santa Monica, CA (US); Gregg Luconi, Monrovia, CA (US)

(73) Assignee: Ocular Data Systems, Inc., Pasadena, CA (US)

(21) Appl. No.: 15/792,685

(22) Filed: Oct. 24, 2017

Related U.S. Application Data
(60) Provisional application No. 62/414,541, filed on Oct. 28, 2016.

(51) Int. Cl. G06F 3/01; A61B 3/113; A61B 3/14

(52) U.S. Cl. CPC G06F 3/013; A61B 3/14; A61B 3/113

(57) ABSTRACT

The image-based system is used to observe and document eye responses, which include eye gaze tracking, pupil size and reactivity, for either one or both eyes sequentially or simultaneously as well as eye lid movements, condition of the sclera, etc. that are recorded and evaluated by police officers. The system includes a faceplate against which the subject places their face. The faceplate can have illumination devices mounted thereon which transmit beams of infrared light and visual light to illuminate the subject's eyes. An imaging device is removably mounted on the frame of the image-based system to observe and document eye responses to generate images of the subject's illuminated eyes. The generated images are also transmitted to an image display device for display to a test administrator.

### CROSS-REFERENCE TO RELATED APPLICATIONS

The present application claims priority to and the benefit of the filing date of U.S. Provisional Patent Application No. 62/414,541 filed Oct. 28, 2016 and titled “Image-Based System, Having Interchangeable Vision Elements, To Observe And Document Eye Responses,” the entire contents of which are hereby incorporated herein

### FIELD OF THE INVENTION

This apparatus can be used both as a police body camera and as an impairment measurement tool to implement the observation and measurement of a subject's eye gaze tracking, pupil size and reactivity as well as monitoring the subject's eye behavior.

### BACKGROUND OF THE INVENTION

It is a problem to determine whether a subject is impaired (either from the use of alcohol or drugs, sleep deprivation and/or other medical condition). This impairment determination can be reliably accomplished by the measurement of eye gaze tracking and monitoring eye behavior (e.g., pupil size and reactivity, blink rate, gaze, etc.).

The field of use of this system is primarily law enforcement, used by officers for testing individuals in the field who are stopped for suspicion of Driving Under the Influence (DUI) or Driving While Impaired (DWI), and this system can also find applications in the workplace where worker safety is of importance as well as that of their co-workers and the general public.

Prior impairment determination systems, such as those disclosed in U.S. Pat. No 7,357,507, titled Image-Based System to Observe and Document Eye Responses and U.S. Pat. No 7,338,166, titled Image-Based System To Observe And Document Eye Responses Having A Reflective Protractor For Measurement Of Stimulus Position, teach the use of a headset that has a faceplate against which the subject places their face. Illumination devices transmit beams of infrared and/or white light, over the faceplate or through one or more openings to illuminate the subject's eyes. One or more imaging devices are mounted on the headset opposite the eyes to generate images of the subject's illuminated eyes. The generated images are transmitted to an image recording device for storage and to an image display device for display to a test administrator. This architecture is embodied in the commercially available image-based system to observe and document eye responses termed “DAX™ Evidence Recorder” produced by Ocular Data Systems, Inc., located in Pasadena, Calif.

More and more, the patrol officer is typically equipped with a Body Worn Camera (body cam), wireless radio, optional dash-mounted camera and smartphone and the above-noted image-based Evidence Recorder to provide communication capability with the dispatch office, data recording of an encounter with a suspect, and on-site accurate measurement of the impairment of the subject. A body cam is a video recording system that is typically utilized by law enforcement officers to record their interactions with the public and/or gather video evidence at crime scenes, and has been known to increase both officer and citizen accountability. Body cams are notable because their placement, often on the front of the police officer's shirt, provides for first-person perspective and a more complete chain of evidence.

Furthermore, the above-noted multitude of data producing elements may each have their own unique communication and data format and data storage requirements, further complicating the administration of law enforcement. Thus, there is a need to simplify the equipment needs of the patrol officer without negatively impacting the quality and thoroughness of the data generation and collection.

### BRIEF SUMMARY OF THE INVENTION

The above described problems are solved and a technical advance achieved by the present Image-Based System, Having Interchangeable Elements, To Observe And Document Eye Responses (termed “image-based system to observe and document eye responses” herein) which multi-purposes the existing body cam for use both as a body camera and as the image generation element in an impairment measurement system.

Based on the technology and algorithms taught by prior art U.S. Pat. No. 7,357,507, titled “Image-Based System to Observe and Document Eye Responses” and U.S. Pat. No 7,338,166, titled “Image-Based System To Observe And Document Eye Responses Having A Reflective Protractor For Measurement Of Stimulus Position” and U.S. Pat. No. 7,614,745 titled “System for Analyzing Eye Responses To Automatically Determine Impairment of a Subject”, one embodiment of an image-based system to observe and document eye responses is disclosed herein that can be used to implement the procedures defined in the Standardized Field Sobriety Test (SFST), the Advanced Roadside Impaired Driving Enforcement (ARIDE) test, as well as the Drug Recognition Expert (DRE) Program as developed by the National Highway Traffic Safety Administration (NHTSA) with certified training under the auspices of the International Association of Chiefs of Police (IACP). These eye responses may include gaze tracking, pupil size, pupil reactivity, for either one or both eyes (sequentially or simultaneously) as well as eye lid movements, condition of the sclera, etc. that are recorded and evaluated by police officers. The system can include a faceplate against which the subject places their face. The system typically has illumination devices mounted thereon which transmit beams of infrared light and visual light to illuminate the subject's eyes. An imaging device is mounted on the frame of the image-based system to observe and document eye responses to generate images of the subject's illuminated eyes. The generated images are transmitted to an image recording device for storage and to a display device for display to a test administrator.

The present embodiment of the image-based system to observe and document eye responses has an interchangeable camera module as the imaging device such that the ‘housing’ could accept certain law enforcement ‘body cams’ including those from other manufacturers in order to give a more ubiquitous application for those companies that may want to incorporate the specialized application into their product line. Alternatively, the police officer can use a camera equipped cell phone or a body cam that does not have a rectangular form factor as shown herein. The image-based system to observe and document eye responses includes a faceplate used for testing eye responses that includes one or more of the following:

Matched optics that receive light from LEDs in the faceplate (visible and/or infrared and or other wavelengths) that illuminate the subject's eyes;

Matched optics that focuses the image of the subject's eyes onto the lenses/optics of a body cam; and

The body cam and the faceplate may communicate through one or more channels, including a wired connector, a wireless connection utilizing RF and or IR, and or acoustic or other method of one or two-way data and control information.

### BRIEF DESCRIPTION OF THE DRAWINGS

FIG. 1 illustrates a perspective view of the present image-based system to observe and document eye responses;

FIG. 2 illustrates a perspective view of the camera insertion mechanism of the present image-based system to observe and document eye responses;

FIGS. 3 and 4 illustrate a side view of the modular faceplate mechanism of the present image-based system to observe and document eye responses;

FIG. 5 illustrates a side view of the removable camera cover mechanism of the present image-based system to observe and document eye responses;

FIG. 6 illustrates a block diagram of the electrical interconnections of the present image-based system to observe and document eye responses;

FIG. 7 illustrates a side view of a police officer administering the Standardized Field Sobriety Test;

FIG. 8 illustrates a rear perspective view of the present image-based system to observe and document eye responses; and

FIGS. 9A and 9B illustrate, respectively, a subject's eyes when just the IR LED's are powered on, i.e. testing in darkness; and the subject's eyes with the IR LED's on, and the left visible spectrum LED on.

### DETAILED DESCRIPTION OF THE INVENTION

In determining whether a subject is impaired (no matter the cause) the monitoring of eye behavior (e.g., blink rate, gaze, etc.) can be used.

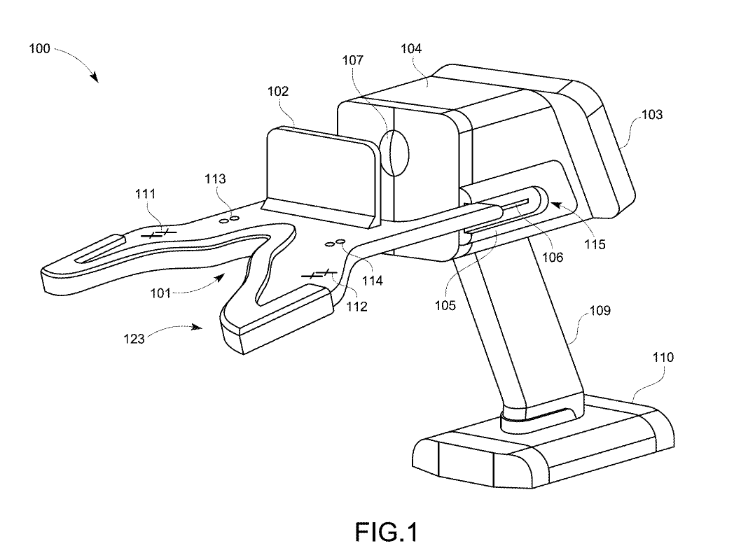

FIG. 1 illustrates a perspective view of one embodiment of the present image-based system to observe and document eye responses, where an interchangeable camera module includes a ‘housing’ that can accept certain law enforcement ‘body cams’ in order to give a more ubiquitous application for those companies that may want to incorporate the specialized application into their product line of body cams; and, for officers and agencies to extend the utility of their existing cameras, or reduce costs, or reduce the number of cameras an officer carries, such as for motorcycle officers. Whereas in-car video cameras record what can be seen from an officer's car, body cams what is happening when an officer is engaged in duties outside of the car in order to supplement or expand on officer's activity when she/he is not visible to or in the range of the in-car camera. The defined functions of the body cam are to record evidence of activities and behaviors relevant to a crime, deter violence or negative behavior against an officer, and improve the accountability of patrol officers to thereby reduce the number of complaints against officers. Body cams are mobile audio and video capture devices that allow officers to record what they see and hear. They can be attached to various body areas, including the head; by the helmet, glasses, or other means; or to the body by means of the pocket, badge or other means of attachment. In purchasing such a system, requirements and trade-offs are dependent on the intended use, budget, unit coast, interoperability, operating environment, etc. Specifications to consider include battery life, video quality, recording limits, night recording, camera focal width, audio recording, camera placement, and radio integration capability. Although most, if not all, body cams have internal memory—or memory that is instantly transmitted to a module in the police car, motor cycle, the “Cloud”, etc., some future technology may require a ‘separate’ memory in the image-based system to observe and document eye responses from what might be lacking in a body cam.

Being able to use the existing body cam and interface it to the image-based system to observe and document eye responses is a benefit in terms of a reduction of the cost of goods for having a single camera do more than one application. The concept is to have an image-based system to observe and document eye responses that could accept the attachment of approved body cams, which come in various configurations. The faceplate in this system has certain elements necessary for its specific application, like infrared LED's to illuminate the eyes of the subject (to simulate the ‘dark environment’ required by the DRE protocol, as well as to enhance the observation of the black pupil in dark-iris individuals), appropriate interface lens to make the field of view of the attached body cam appropriate for the close-up view of the eyes of the subject, bright light stimulation for observation of ocular pupil reactivity, etc.

It is anticipated that a data interface, specific to each manufactured body cam device, couples the body cam to the image-based system to observe and document eye responses. The coupling mechanism could range from being a direct wired interface between the image-based system to observe and document eye responses and the body cam, to an interface that uses appropriate fiber optics, or wireless, or any other existing or anticipated means to bring the two elements together. In terms of ‘matched optics’, one aspect of this innovation is to have the body cam lens system, which is typically geared for seeing a ‘wide far away scene’ matched to a lens that would give a close-up, more narrow field of view. These cameras produce a vast amount of data that is either stored on the law enforcement department's server or in a cloud-based storage.

### System Block Diagram

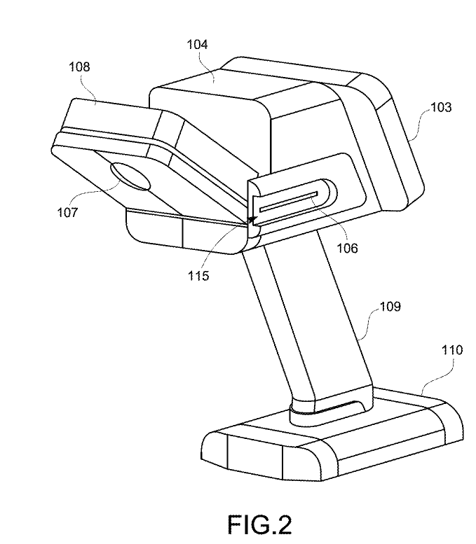

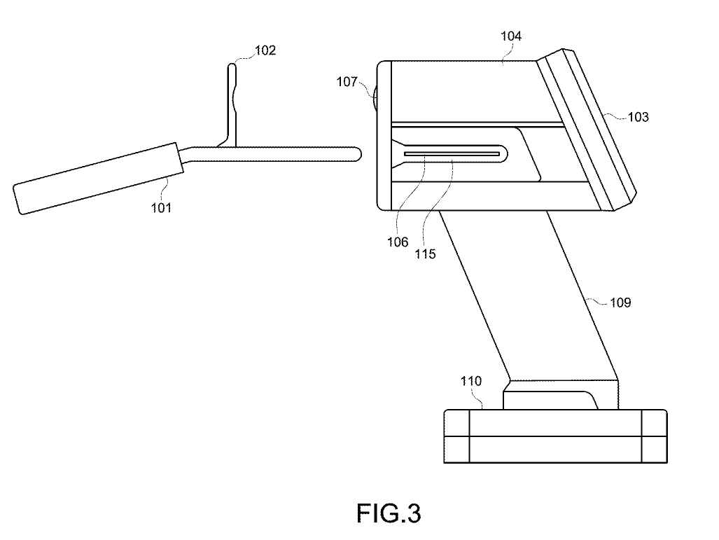

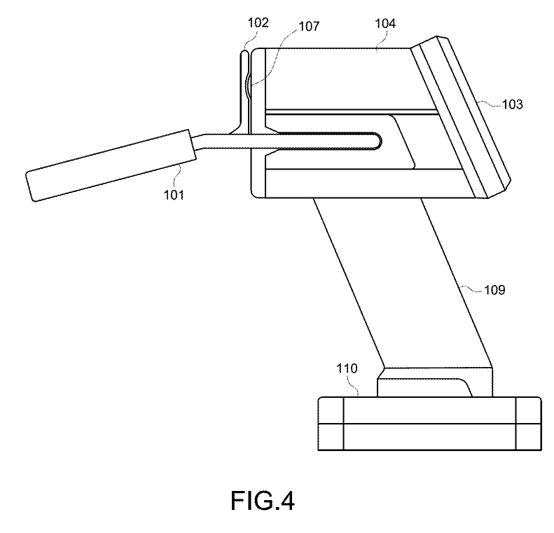

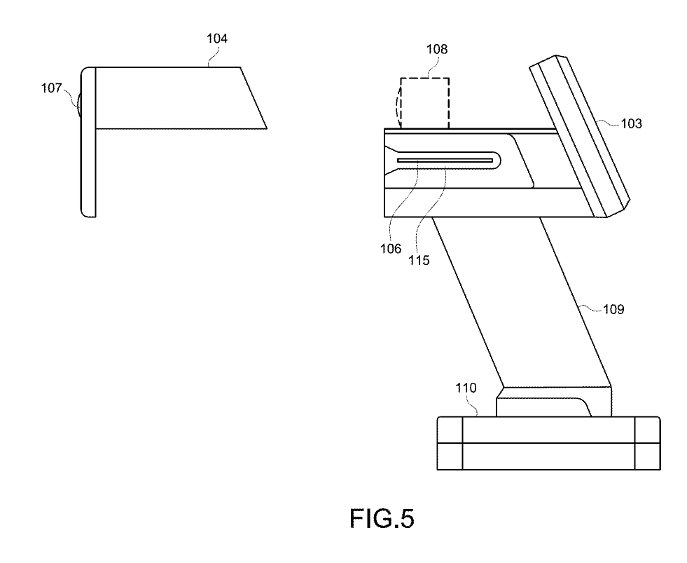

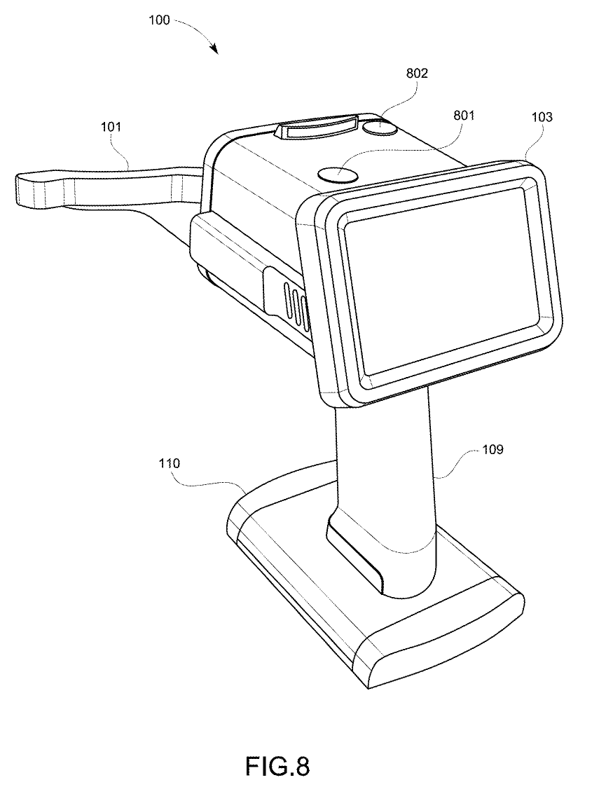

FIG. 1 illustrates perspective view of the present image-based system to observe and document eye responses 100 (with additional details shown in FIG. 2 which illustrates a perspective view of the camera insertion mechanism of the present image-based system to observe and document eye responses; FIGS. 3 and 4 which illustrate a side view of the faceplate mechanism of the present image-based system to observe and document eye responses; and FIG. 5 which illustrates a side view of the present image-based system with a display screen and FIG. 8 illustrates a rear perspective view of the present image-based system to observe and document eye responses).

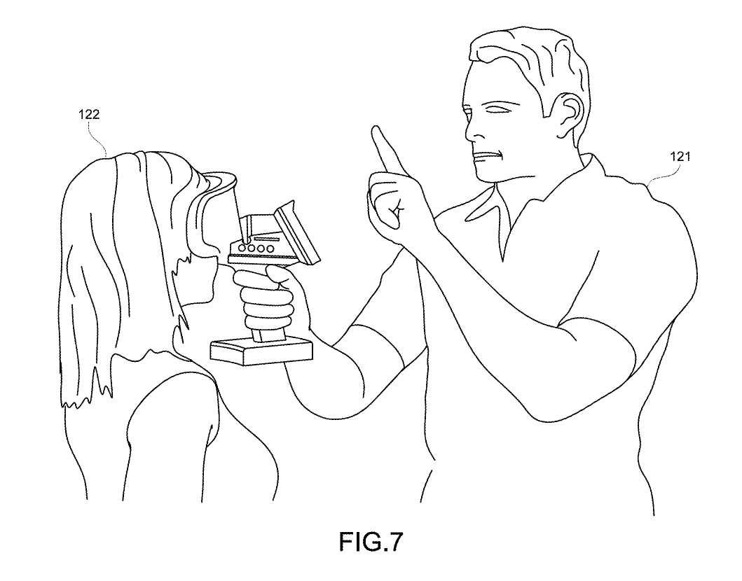

The present image-based system to observe and document eye responses 100 is used to administer impairment tests, such as the Drug Recognition Expert examination, ARIDE test and/or the Standardized Field Sobriety Test. A faceplate 101, attachable to frame 105, is used to administer the test to the subject 122 (shown in FIG. 7) and it includes illumination devices 111-114 that are attached to faceplate 101, which includes a slot 123 to position the subject's face with respect to illumination devices 111-114. A source of electrical power, such as batteries 116, is provided to power illumination devices 111-114 and display device 103, with the batteries typically located in the base 110 of the grip 109 of frame 105.

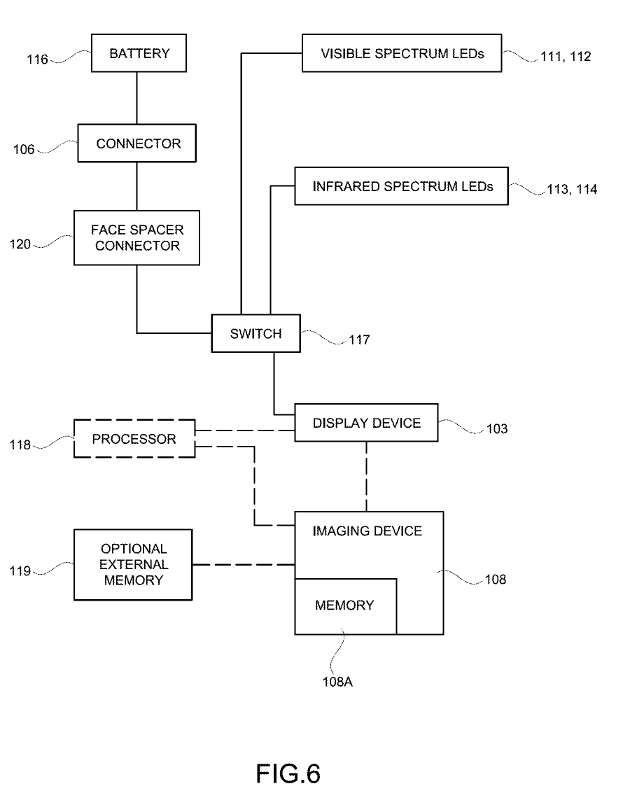

This system to observe and document eye responses 100 also allows for playback of the eye responses and associated audio for review and training purposes, since the image output is recorded in real time. Regarding the electrical contacts 106, they are mounted along the inside of slot 115 and engage matching contacts 120 mounted along the inside of the prongs on the faceplate 101 when the faceplate 101 slid into slot 115 formed in frame 105 that allows these contacts to pass power to the illumination devices 111-114 and display device 103 when the faceplate 101 is slid into the frame 105. As shown in FIG. 6, the batteries 116 are typically interconnected to the electrically powered elements of the image-based system to observe and document eye responses 100 via connector 106. Thus, the visible spectrum LEDs 111, 112; the infrared spectrum LEDs 113, 114 as well as the image display device 103 are all interconnected and powered when the faceplate is inserted into slot 115. Alternatively, a power on/off switch 117 can be used to controllably power the above-mentioned elements under the control of the patrol officer 121 as described below.

An imaging device 108 (body cam) stores the generated image data and can optionally be connected to an image recording device 119 for capturing and storing image data. The interconnection between the imaging device 108 and an image recording device 119 can be wired or wireless, using a cable or wireless communication protocol, respectively. The imaging device 108 is typically activated by a power on/off switch 801 and recording is controlled via switch 802, which are typically part of the imaging device 108. A display device 103 is also provided to present a real time representation to a test administrator 121 (FIG. 7) of the image generated by the imaging device 108 and stored in body cam memory 108A, and the imaging device 108 typically is optionally connected to the image recording device 119 via a wired or wireless connection. One or more lenses 102 are also attached to faceplate 101 to translate the view of the subject's eyes presented to imaging device 108. In particular, matched optics 102, 107, located in an image transmission path from the subject's illuminated eyes to the imaging device 108, receives light from the LEDs 111-114 mounted on the faceplate 101 (visible and or infrared and or other wavelengths) that are used to illuminate the subject's eyes (or the subject's eyes in ambient light to provide a baseline). The matched optics focuses the image of the subject's eyes onto the lenses/optics of an off the shelf body cam (imaging device 108) and/or changes the focal length, depth of field, angle, etc.

### Light Emitting Diodes

The faceplate 101 has two types of illumination devices 111-114 which transmit beams of light to illuminate the subject's eyes. The use of infrared illumination enhances the observation, as well as the documentation and review of the eye movement and pupil responses, on the DAX video screen or any other monitor linked (i.e. HDMI, WiFi, etc.) to the DAX especially in those individuals with dark pigmented irises. The infrared illumination devices 111, 112 are utilized to provide invisible illumination of the subject's eyes for those portions of the law enforcement Drug Recognition Expert exam, AIRIDE test and/or the Standardized Field Sobriety Test that requires pupil size determination in what is called a ‘dark room’ test. This portion of the exam has the law officer 121 have the suspect in a dark or very dimly lit room in order to assess the subject's pupil size in a dark environment. In normal individuals, the pupil would dilate, but subjects under the influence of narcotics (opioids, heroin, etc.) would present a pupil that stays constricted, i.e. pinpoint pupils. An easy indicator of this type of impairment.

In order to allow video recording during a ‘dark room’ test, the image-based system to observe and document eye responses 100 has this invisible infrared illumination which does not stimulate the pupil but allows the camera to image the eye (as the cameras are sensitive to this invisible infrared illumination) with the image available on the image-based system to observe and document eye responses 100 screen. An optional system cover and/or a switch to turn off the one or more displays is also available to further control the amount of ambient light during the examinations. The IR lighting also causes an increase in contrast relative to the black pupil on the display device 103 thus increasing contrast with the pupil thus aiding in determination of pupil size. The test administrator 121 is able to view the subject's eye(s) while providing the stimulus target (finger, pen tip, etc.) for the subject to view. The subject's eye responses are displayed on a display device 103. The display device 103 is powered via the electrical interconnection of batteries 116 with faceplate 101 via connector 106 or can be activated via the use of a switch 117 once the faceplate 101 is connected to batteries 116.

The law enforcement exam requires the determination of the suspect's pupil reaction to various levels of visible illumination. In the field, the law officer typically has a small pocket pen light that they use to illuminate the eye, similar to what a physician might do in their office. In contrast, the image-based system to observe and document eye responses 100 has positioned the visible spectrum LEDs 113, 114 to illuminate the suspect's eyes per the recommended impairment testing procedure. They are turned on by the patrol officer at the appropriate time during the exam. Thus, to provide the patrol officer with control over the activation of the two sets of LEDs, a multi-position switch 117, typically mounted on the handle of the frame 105, can be provided to power on the LEDs and allow for individual control of the IR LEDs 111, 112 and the visible spectrum LEDs 113, 114. It is conceivable that the control of the visible illumination could be voice controlled as well.

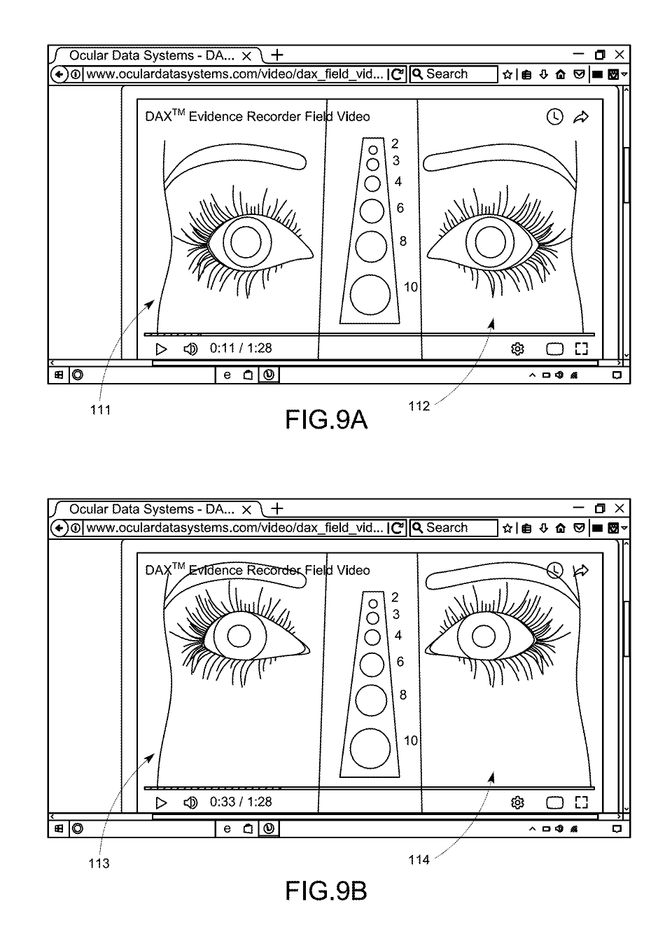

In one configuration, the IR LED's are ‘ON’ all the time. When the patrol officer wants to use the visible light, he can choose either the Right or Left visible spectrum LED. FIGS. 9A and 9B illustrate, respectively, a subject's eyes when just the IR LED's are powered on, i.e. testing in darkness; and the subject's eyes with the IR LED's on, and the left visible spectrum LED on. When just the IR LED's 111, 112 are powered on, the subject's eyes are dilated as shown in FIG. 9A and once the patrol officer activates one or both visible spectrum LEDs 113, 114 the subject's eyes respond to the presence of visible light and the irises constrict as shown in FIG. 9B. The body cam 108 records the voices of the patrol officer and the subject to provide a multi-media context of the impairment test being administered.

A further enhancement of functionality of the image-based system to observe and document eye responses 100 is to provide processor 118 which is disclosed in U.S. Pat. No. 7,614,745 and that executes an automated analysis of the images that are generated by the imaging device 108 to automatically determine impairment of the subject. The determination of impairment, using the generated images, is presented to the law enforcement officer in real time to enable them to make an immediate evaluation of the impairment of the subject. The data interconnections among the elements of the image-based system to observe and document eye responses 100 are shown in FIG. 6 by dotted lines interconnecting these elements.

### Additional Capabilities

One possible expansion of the capability of this system is to add a breath analyzer (active or passive) so that eyes as well as breath alcohol measurements or a marijuana breath test, etc. is used as an additional or integrated capability. The breath analyzer (not shown) can be mounted on the frame 105 of the image-based system to observe and document eye responses 100 below the location of the faceplate 101, when it is installed. The subject can provide a breath sample into the breath analyzer as their eye responses are measured and documented. Another idea is adding a camera to existing penlights, which the patrol officers use now to provide the ‘target’ that the suspect looks at when they do their testing.

Other possibly capabilities would be, but not limited to:

The use of iris color, as recorded by the imaging device 108 and displayed on display device 103, as an additional biometric for identifying the individual being tested.

Using the temperature of the eye as detected from a sensor that is able to determine such, to compare the individuals normal body temperature as an additional metric for determining a change in the subject's physiology, related to impairment and the testing.

Using special lens/sensors and/or wavelength illumination changes to determine blood flow characteristics from the eye which may be important in the screening of an individual for impairment.

Using the same or similar special lens/sensor and/or wavelength illumination changes to determine blood oxygen levels from the eye which may be important in the screening of an individual for impairment.

Using the same or similar special lens/sensor and/or wavelength illumination changes to determine the color of the sclera (the white part of the eye beyond the outer boundary of the iris as certain impairing agents causes dilation of the sclera blood vessels giving the it a more reddish hue.

### SUMMARY

The image-based system to observe and document eye responses can be used to implement the procedures such as those defined in the Standardized Field Sobriety Test ARlDE and/or the Drug Recognition Expert Program. These eye responses include pupil movement, size and responses, for either one or both eyes simultaneously as well as eye lid movements, condition of the sclera, etc. that are recorded and evaluated by police officers. The system includes a faceplate against which the subject places their face. The faceplate has illumination devices which transmit beams of infrared light and visual light to illuminate the subject's eyes. One or more imaging devices are mounted on the frame to generate images of the subject's illuminated eyes. The generated images are transmitted to an image recording device for storage and one or more image display devices for display to a test administrator.

What is claimed:

1. An image-based system to enable an individual administering an impairment detection test to a subject to observe and document eye responses of the subject to applied visual stimuli, comprising:
a faceplate, removably attached to a frame, shaped to substantially conform to the subject's face when the subject places their face against the faceplate;
at least one illumination device to illuminate the subject's eyes in executing an impairment detection test when the subject places their face against the faceplate; and
at least one imaging device, removably attached to the frame and aligned with the subject's eyes to generate an image of the subject's illuminated eyes when the subject places their face against the faceplate.

2. The image-based system to observe and document eye responses of claim 1 further comprising:
at least one lens, located in an image transmission path from the subject's illuminated eyes to the imaging device, for focusing the image of the subject's eyes onto optics of the imaging device, wherein the at least one lens is attached to the faceplate.

3. The image-based system to observe and document eye responses of claim 1 wherein the frame comprises:
a plurality of electrical contacts which are connected to a source of electrical power and/or used as a data communication channel.

4. The image-based system to observe and document eye responses of claim 3 wherein the faceplate comprises:
a plurality of electrical contacts which engage with the plurality of electrical contacts mounted on the frame to provide electrical power for the at least one illumination device.

5. The image-based system to observe and document eye responses of claim 3 wherein the faceplate comprises:
a plurality of electrical contacts which engage with the plurality of electrical contacts mounted on the frame to provide electrical power for the image display.

6. The image-based system to observe and document eye responses of claim 1 wherein the at least one illumination device comprises:
at least one infrared illumination device which provides invisible illumination of the subject's eyes to enable pupil size determination.

7. The image-based system to observe and document eye responses of claim 1 wherein the at least one illumination device comprises:
at least one visible spectrum illumination device to test a suspect's pupil reaction to various levels of visible illumination.

8. The image-based system to observe and document eye responses of claim 1 further comprising:
at least one display device mounted on the frame and positioned in front of an individual administering the test to display the generated images of a subject's eyes.

9. The image-based system to observe and document eye responses of claim 8 wherein the imaging device communicates with the display device to provide the generated images of a subject's eyes to the display device for presentation to an individual administering the test.

10. The image-based system to observe and document eye responses of claim 1 wherein the imaging device includes memory to record the generated images of a subject's eyes.

11. The image-based system to observe and document eye responses of claim 1 further comprising:
a processor executing software that controls the at least one illumination device to execute at least one impairment detection test of the subject's optical responses to determine whether the subject is impaired based on the images generated by the imaging device in response to the at least one test of the subject's optical responses.

12. An image-based system to enable an individual administering an impairment detection test to a subject to observe and document eye responses of the subject to applied visual stimuli, comprising:
a faceplate, removably attached to a frame, shaped to substantially conform to a subject's face when the subject places their face against the faceplate;
at least one illumination device attached to the faceplate to illuminate the subject's eyes in executing an impairment detection test when the subject places their face against the faceplate;
at least one imaging device, removably attached to the frame and aligned with the subject's eyes to generate an image of a subject's eyes when the subject places their face against the faceplate; and
at least one lens, located in an image transmission path from the subject's illuminated eyes to the imaging device, for focusing the image of the subject's eyes onto optics of the imaging device; and
at least one display device mounted on the frame and positioned in front of the individual administering the impairment detection test to display the generated images of the subject's eyes.

13. The image-based system to observe and document eye responses of claim 12 further comprising:
a switch connected to a source of electrical power for controllably applying electrical power to the imaging device.

14. The image-based system to observe and document eye responses of claim 13 further comprising:
wherein the frame comprises:
a plurality of electrical contacts which are connected to a source of electrical power via the switch;
wherein the faceplate comprises:
a plurality of electrical contacts which engage with the plurality of electrical contacts mounted on the frame to provide electrical power for the at least one illumination device;

15. The image-based system to observe and document eye responses of claim 12 wherein the at least one illumination device comprises:
at least one infrared illumination device which provide invisible illumination of the subject's eyes to enable pupil size determination.

16. The image-based system to observe and document eye responses of claim 12 wherein the at least one illumination device comprises:
at least one visible spectrum illumination device to test a suspect's pupil reaction to various levels of visible illumination.

17. The image-based system to observe and document eye responses of claim 12 wherein the imaging device communicates with the display device to provide the generated images of a subject's eyes to the display device for presentation to an individual administering the test.

18. The image-based system to observe and document eye responses of claim 12 wherein the imaging device includes memory to record the generated an image of a subject's eyes.

19. The image-based system to observe and document eye responses of claim 12 further comprising:
a processor executing software that controls the at least one illumination device to execute at least one impairment detection test of the subject's optical responses to determine whether the subject is impaired based on the images generated by the imaging device in response to the at least one test of the subject's optical responses.
